(19) United States
(12) Patent Application Publication
(10) Pub. No.: US 2010/0324034 A1
(43) Pub. Date: Dec. 23, 2010

(54) Methods of Using SAHA for Treating HIV Infection

(76) Inventors: Daria J. Hazuda, Doylestown, PA (US); Amy S. Espeseth, Chalfont, PA (US); David M. Margolis, Chapel Hill, NC (US); Nancie M. Archin, Chapel Hill, NC (US)

Correspondence Address: MINTZ LEVIN COHN FERRIS GLOVSKY & POPEO, ONE FINANCIAL CENTER, BOSTON, MA, 02111, US

(21) Appl. No.: 12/526,437

(22) PCT Filed: Feb. 8, 2008
(86) PCT No.: PCT/US08/01738

§ 371 Date: Aug. 7, 2009

Related U.S. Application Data
(60) Provisional application No. 60/900,558, filed on Feb. 8, 2007.

(51) Int. Cl. A61K 31/167; A61P 31/18; A61K 31/536; A61K 31/496; A61K 31/513

(52) U.S. Cl. 514/230.5; 514/575; 514/253.01; 514/269

(57) ABSTRACT

The present invention relates to pharmaceutical preparations and methods for treating individuals infected with the human immunodeficiency virus (HIV). The pharmaceutical preparations comprise SAHA and another anti-viral agent. The invention also relates to methods for treating HIV infected patients, particularly patients with persistent, latent HIV infection of CD4⁺ T cells, and thus make it possible to not just suppress but to eradicate the HIV infection.

### FIELD OF THE INVENTION

The present invention relates in part to a method of treating HIV infection by administering a histone deacetylase (HDAC) inhibitor, e.g., SAHA, in combination with one or more anti-retroviral agent(s).

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight life-threatening opportunistic infections. Statistics (UNAIDS: Report on the Global HIV/AIDS Epidemic, December 1998), indicated that as many as 33 million people worldwide were infected with the virus in 1998. In addition to the large number of individuals already infected, the virus continues to spread. Estimates from 2005 point to close to 6 million new infections in that year alone. In that same year. there were approximately 2.5 million deaths associated with HIV and AIDS.

Highly active antiretroviral therapy (HAART) has been used to effectively suppress replication of HIV (Gulick et al. (1997) N. Engl. J. Med. 337:734-9; Hammer et al. (1997) N. Engl. J. Med. 337:725-733). However, HAART is primarily efficacious with regard to the prevention of the spread of infection into uninfected cells and this therapy does not efficiently reduce the residual, latent proviral DNA integrated into the host cellular genome (Wong et al. (1997) Science 278:1291-1295; Finzi et al. (1997) Science 278:1295-1300 (see comments); Finzi et al. (1999) Nat. Med. 5:512-517; Zhang et al. (1999) N. Engl. J. Med. 340:1605-1613). HIV will remain a chronic viral infection unless there are therapeutic treatments for persistent, latent infection of resting CD4⁺ T cells.

Mechanisms that allow HIV to establish latency are unknown. However, local chromatin effects have long been thought to contribute to the durable suppression of HIV proviral expression, and latently infected cells recovered from a T lymphocyte cell line infected in vitro were found to contain HIV integrated in or close to alphoid repeat elements in heterochromatin (Jordan et al., (2003) EMBO J, 22:1868-1877; Winslow et al. (1993) 196: 849-854). Furthermore, evidence suggests that modulation of histone architecture within the viral promoter participates in the establishment of the viral latency. Specifically, the integrated HIV long terminal repeat (LTR) promoter requires remodeling to allow expression, and histone acetylation to respond to NF-kB activation (van Lint C et al., (1995) EMBO J., 15:1112-1120; Sheridan et al., Genes Dev 11:3327-3340 (1997); El Kharroubi et al., Mol Cell Biol 18:2535-2544 (1998)).

There is a need in the art for interventions that derepress the LTR and result in selective expression of quiescent HIV. Activation of viral gene expression in the presence of HAART is expected to lead to T-cell apoptosis or destruction of a cell expressing viral proteins by the host immune system, and thus depletion of the reservoir of persistent HIV infection. These much needed interventions would make it possible to not just suppress but to further eradicate the HIV infection.

### SUMMARY OF THE INVENTION

The present invention relates to pharmaceutical preparations and methods for treating HIV infection. The present invention is based, in part, on the discovery that histone deacetylase (HDAC) inhibitors, for example suberoylanilide hydroxamic acid (SAHA), can be used to activate the expression of quiescent, integrated HIV within resting CD4⁺ T cells, thereby depleting latent HIV infection. Further, selective induction of latent infection would allow antiretroviral drugs and the antiviral immune response to access and eradicate residual HIV infection.

In one aspect, the invention relates to a method of treating HIV infection in a subject comprising administering SAHA or a pharmaceutically acceptable salt or hydrate thereof and one or more anti-retroviral agents. The invention also encompasses a method of treating latent HIV infection in a subject comprising administering SAHA or a pharmaceutically acceptable salt or hydrate thereof and one or more anti-retroviral agents. In certain embodiments, SAHA is administered with one anti-retroviral agent. In other embodiments, SAHA is administered with two anti-retroviral agents. SAHA can also be administered with three, four, five, or any additional number of anti-retroviral agents as part of a multi-drug “cocktail.” Other aspects of the invention relate to a method of depleting latent HIV infection within resting CD4⁺ T cells, and a method of activating expression from the HIV long terminal repeat (LTR) promoter with an effective amount of SAHA.

The methods of the present invention include administration of SAHA in combination with at least one anti-retroviral agent to a subject infected with HIV. In one embodiment of the invention, SAHA is administered to a subject infected with HIV in combination with a reverse transcriptase inhibitor. In another aspect of the invention, SAHA is administered to a subject infected with HIV in combination with a nucleoside reverse transcriptase inhibitor. In yet another aspect of the invention, SAHA is administered to a subject infected with HIV in combination with a non-nucleoside reverse transcriptase inhibitor. In other aspects, SAHA is administered to a subject infected with HIV in combination with a protease inhibitor. In another aspect, SAHA is administered to a subject infected with HIV in combination with fusion inhibitors. In another aspect of the invention, SAHA is administered to a subject infected with HIV in combination with entry inhibitors. SAHA may also be administered to a subject infected with HIV with intensified antiretroviral therapy, in which an existing cocktail of drugs is increased by the addition of an antiretroviral drug that targets a different viral enzyme. For example, SAHA may be administered with at least one nucleoside reverse transcriptase inhibitor and one non-nucleoside reverse transcriptase inhibitor. In some embodiments, SAHA may be administered with two nucleoside reverse transcriptase inhibitors and one non-nucleoside reverse transcriptase inhibitor. Further, a protease inhibitor, a fusion inhibitor and an integrase inhibitor may be added to the anti-retroviral cocktail.

Suitable anti-retroviral agents for use in the therapeutic compositions and methods described herein include nucleoside reverse transcriptase inhibitors, non-nucleoside reverse transcriptase inhibitors, protease inhibitors, co-receptor antagonists, retroviral integrase inhibitors, viral adsorption inhibitors, viral specific transcription inhibitors, and cyclin dependent kinase inhibitors. In one embodiment, the anti-retroviral agent is selected from the group consisting of efavirenz, indinavir sulfate, and raltegravir potassium. In a preferred embodiment, the anti-retroviral agent is raltegravir potassium.

In further embodiments, the anti-retroviral agent can be administered for a specific duration, and during that period, SAHA is administered concurrently, simultaneously, sequentially in any order, or at specific intervals. For example, the anti-retroviral agent and SAHA can be administered on the same or adjacent day, sequentially, simultaneously, alternating or consecutively in any order during the course of anti-retroviral therapy or regimen.

In one aspect of the invention, the administration of SAHA and the administration of anti-retroviral agent are performed concurrently. In the preferred embodiment of the present invention, SAHA is administered orally.

A further aspect of the invention relates to pharmaceutical compositions useful for treatment of HIV infection comprising SAHA, an amount of one or more anti-retroviral agent selected from the group consisting of a nucleoside reverse transcriptase inhibitor, a non-nucleoside reverse transcriptase inhibitor, a protease inhibitor, a fusion inhibitor, an entry inhibitor, an integrase inhibitor, a co-receptor antagonist, a viral adsorption inhibitor, a viral specific transcription inhibitor, and a cyclin dependent kinase inhibitor and a combination thereof and a pharmaceutically acceptable carrier.

Another aspect of the invention relates to pharmaceutical compositions useful for treatment of HIV infection comprising SAHA, an amount of one or more anti-retroviral agents selected from the group consisting of a non-nucleoside reverse transcriptase inhibitor, a protease inhibitor, an integrase inhibitor, and a combination thereof, and a pharmaceutically acceptable carrier.

A further aspect of the present invention relates to pharmaceutical compositions useful for treatment of HIV infection comprising SAHA, an amount of one or more anti-retroviral agents selected from the group consisting of efavirenz, indinavir sulfate and raltegravir potassium and a pharmaceutically acceptable carrier.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and are encompassed by the following detailed description and claims.

### BRIEF DESCRIPTION OF THE DRAWINGS

The foregoing and other objects, features and advantages of the invention will be apparent from the following more particular description of various embodiments of the invention, as illustrated in the accompanying drawings.

FIG. 1 is a graph depicting dose response curve for SAHA activating expression of quiescent, integrated HIV in the ACH-2 cell line.

FIG. 2A: is a flow diagram of the experimental methods described further in Example 3.

FIG. 2B: depicts the results of flow cytometry, detecting CD3 and CD28 cell surface expression in a primary cell assay for HIV latency in the presence or absence of the integrase inhibitor L-810.

FIG. 3: is a bar graph showing a comparative study of a number of different HDAC inhibitors and their ability to induce HIV expression from latently infected resting cells. The columns for % p24 induction appear sequentially from left to right in the order of spinoculation, wherein the bar at the far left represents spinoculation with no virus and the striped bar at the far right represents spinoculation in the presence of 5 mM HMBA.

### DETAILED DESCRIPTION OF THE INVENTION

Methods and compositions are provided for treating patients infected with HIV. The compositions and methods of the present invention resolve the shortcomings of current HIV therapies by achieving selective expression of the quiescent HIV in the presence of anti-retroviral therapy and thus depleting the reservoir of persistent HIV infection, and making it possible to not just suppress, but to eradicate HIV.

Specifically, the methods of the instant invention relate to treatment of HIV infection in a subject comprising administering SAHA or a pharmaceutically acceptable salt or hydrate thereof, in combination with one or more anti-retroviral agents. The methods of the instant invention further relate to depleting latent HIV infection within resting CD4⁺ T cells. The methods of the instant invention also relate to activating expression from the HIV long terminal repeat (LTR) promoter with an effective amount of SAHA.

The methods of the present invention also include administration of an effective amount of SAHA in combination with at least one anti-retroviral agent wherein the retroviral agent may be selected from the group comprising nucleoside reverse transcriptase inhibitors, non-nucleoside reverse transcriptase inhibitors, protease inhibitors, co-receptor antagonists, retroviral integrase inhibitors, viral adsorption inhibitors, viral specific transcription inhibitors, cyclin dependent kinase inhibitors, and combinations thereof. The “effective amount of SAHA” is an amount to selectively activate expression of quiescent HIV infection within resting CD4⁺ T cells and less than an amount with causes toxicity in a patient.

### DEFINITIONS

The term “treating” in its various grammatical forms in relation to the present invention refers to curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease. Because some of the inventive methods involve the physical removal of the etiological agent, the artisan will recognize that they are equally effective in situations where the inventive compound is administered prior to, or simultaneous with, exposure to the etiological agent and situations where the inventive compounds are administered after (even well after) exposure to the etiological agent.

The term “preventing” in the context of the present invention means that the effects of a disease state or disease causative agent has been obviated due to administration of an agent, such as those disclosed herein. A similar term in this context is “prophylaxis.”

As recited herein, “HDAC inhibitor” (e.g., SAHA) encompasses any synthetic, recombinant, or naturally-occurring inhibitor, including any pharmaceutical salts or hydrates of such inhibitors, and any free acids, free bases, or other free forms of such inhibitors. “Hydroxamic acid derivative,” as used herein, refers to the class of histone deacetylase inhibitors that are hydroxamic acid derivatives. Specific examples of inhibitors are provided herein.

“Patient” or “subject” are used interchangeably herein and refer to the recipient of treatment. Mammalian and non-mammalian subjects are included. In a specific embodiment, the subject is a mammal, such as a human, canine, murine, feline, bovine, ovine, swine, or caprine. In a particular embodiment, the subject is a human. Preferably, a subject is one who has been infected with HIV, but can also encompass those who are at risk of being infected with HIV, or those who lack clinical symptoms of HIV infection, but who nevertheless may be infected with HIV present in cells in a latent form.

The terms “intermittent” or “intermittently” as used herein means stopping and starting at either regular or irregular intervals.

The term “hydrate” includes but is not limited to hemihydrate, monohydrate, dihydrate, trihydrate, and the like.

As used herein, the term “viral infection” describes a diseased state in which a virus invades healthy cells, uses the cell's reproductive machinery to multiply or replicate and ultimately lyse the cell resulting in cell death, release of viral particles and the infection of other cells by the newly produced progeny viruses. Latent infection by certain viruses is also a possible result of viral infection.

As used herein, the term “treating viral infections” means to inhibit the replication of the particular virus, to inhibit viral transmission, and to ameliorate or alleviate the symptoms of the disease caused by the viral infection. The treatment is considered “therapeutic” if there is a reduction in viral load, decrease in mortality and/or morbidity. “Preventing viral infections” means to prevent the virus from establishing itself in the host. A treatment is considered “prophylactic” if the subject is exposed to the virus, but does not become infected with the virus as a result of treatment.

“LTR” means the long terminal repeat, a 713 base pair DNA sequence repeated at the 5′ and 3′ ends of the HIV genome, which consists of the enhancer and promoter regions for gene expression (U3 region), the RNA start site, and untranslated RNA sequences (RU5) such as the genomic repeat and polyadenylation sites.

“Latency” means a concept describing 1) an asymptomatic clinical condition, 2) the dormant state of viral activity within a population of cells, wherein viral production, viral packaging, and host cell lysis does not occur, or occurs at a very low frequency, or 3) the down-regulation or absence of gene expression within an infected cell. “Latent” in the viral context can mean that the viral genome has integrated into the host cell genome without subsequent viral packaging of the viral genome into a viral capsid or other virus structure, which then causes the host cell to lyse, releasing viral particles that are free to infect other cells in the host. “Latency” in the context of the viral life cycle can also refer to a virus' “lysogenic phase.” In contrast, a virus is in the “lytic” phase if the viral genomes are packaged into a capsid or other viral structure, ultimately leading to lysis of the host cell and release of newly packaged viruses into the host.

### Histone Deacetylases and Histone Deacetylase Inhibitors

Histone deacetylases (HDACs) include enzymes that catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation, and/or apoptosis of transformed cells in vitro and inhibit tumor growth in vivo.

HDACs can be divided into three classes based on structural homology. Class I HDACs (HDACs 1, 2, 3, and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors are compounds that are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures that can assay for the accumulation of acetylated histones can be used to determine the HDAC inhibitory activity of compounds of interest. It is understood that compounds that can inhibit histone deacetylase activity can also bind to other substrates and as such can inhibit other biologically active molecules such as enzymes. It is also understood that the compounds of the present invention are capable of inhibiting any of the histone deacetylases set forth above, or any other histone deacetylases.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assay which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92: 1210-1215 (2000); Butler, L M. et al., Cancer Res. 60: 5165-5170 (2000); Richon, V. M. et al., Proc. Natl. Acad. Sci., USA, 95: 3003-3007 (1998); and Yoshida, M. et al., J. Biol. Chem., 265: 17174-17179 (1990).

For example, an enzymatic assay to determine the activity of an HDAC inhibitor compound can be conducted as follows. Briefly, the effect of an HDAC inhibitor compound on affinity purified human epitope-tagged (Flag) HDAC1 can be assayed by incubating the enzyme preparation in the absence of substrate on ice for about 20 minutes with the indicated amount of inhibitor compound. Substrate ([³H]acetyl-labeled murine erythroleukemia cell-derived histone) can be added and the sample can be incubated for 20 minutes at 37° C. in a total volume of 304. The reaction can then be stopped and released acetate can be extracted and the amount of radioactivity release determined by scintillation counting. An alternative assay useful for determining the activity of an HDAC inhibitor compound is the “HDAC Fluorescent Activity Assay; Drug Discovery Kit-AK-500” available from BIOMOL® Research Laboratories, Inc., Plymouth Meeting, Pa.

In vivo studies can be conducted as follows. Animals, for example, mice, can be injected intraperitoneally with an HDAC inhibitor compound. Selected tissues, for example, brain, spleen, liver etc, can be isolated at predetermined times, post administration. Histones can be isolated from tissues essentially as described by Yoshida et al., J. Biol. Chem. 265: 17174-17179 (1990). Equal amounts of histones (about 1 μg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc—H4) and anti-acetylated histone H3 antibody (αAc—H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

Thus, the present invention includes within its broad scope compositions comprising HDAC inhibitors which are 1) hydroxamic acid derivatives; 2) Short-Chain Fatty Acids (SCFA5); 3) cyclic tetrapeptides; 4) benzamides; 5) electrophilic ketones; and/or any other class of compounds capable of inhibiting histone deacetylases, for use in inhibiting histone deacetylase, inducing terminal differentiation, cell growth arrest and/or apoptosis in neoplastic cells, and/or inducing differentiation, cell growth arrest and/or apoptosis of tumor cells in a tumor.

Non-limiting examples of such HDAC inhibitors are set forth below. It is understood that the present invention includes any salts, crystal structures, amorphous structures, hydrates, derivatives, metabolites, stereoisomers, structural isomers, and prodrugs of the HDAC inhibitors described herein.

A. Hydroxamic Acid Derivatives such as Suberoylanilide hydroxamic acid (SAHA) (Richon et al., Proc. Natl. Acad. Sci. USA 95, 3003-3007 (1998)); m-Carboxycinnamic acid bishydroxamide (CBHA) (Richon et al., supra); Pyroxamide; Trichostatin analogues such as Trichostatin A (TSA) and Trichostatin C (Koghe et al. Biochem. Pharmacol. 56: 1359-1364 (1998)); Salicylbishydroxamic acid (Andrews et al., International J. Parasitology 30, 761-768 (2000)); Suberoyl bishydroxamic acid (SBHA) (U.S. Pat. No. 5,608,108); Azelaic bishydroxamic acid (ABHA) (Andrews et al., supra); Azelaic-1-hydroxamate-9-anilide (AAHA) (Qiu et al., Mol. Biol. Cell 11, 2069-2083 (2000)); 6-(3-Chlorophenylureido) carpoic hydroxamic acid (3Cl—UCHA); Oxamflatin [(2E)-5-[3-[(phenylsulfonyl) amino]phenyl]-pent-2-en-4-ynohydroxamic acid] (Kim et al. Oncogene, 18: 2461 2470 (1999)); A-161906, Scriptaid (Su et al. Cancer Research, 60: 3137-3142 (2000)); PXD-101 (Prolifix); LAQ-824; CHAP; MW2796 (Andrews et al., supra); MW2996 (Andrews et al., supra); or any of the hydroxamic acids disclosed in U.S. Pat. Nos. 5,369,108, 5,932,616, 5,700,811, 6,087,367, and 6,511,990.

B. Cyclic Tetrapeptides such as Trapoxin A (TPX)-cyclic tetrapeptide (cyclo-(L-phenylalanyl-L-phenylalanyl-D-pipecolinyl-L-2-amino-8-oxo-9,10-epoxy decanoyl)) (Kijima et al., J. Biol. Chem. 268, 22429-22435 (1993)); FR901228 (FK 228, depsipeptide) (Nakajima et al., Ex. Cell Res. 241, 126-133 (1998)); FR225497 cyclic tetrapeptide (H. Mori et al., PCT Application WO 00/08048 (17 Feb. 2000)); Apicidin cyclic tetrapeptide [cyclo(N—O-methyl-L-tryptophanyl-L-isoleucinyl-D-pipecolinyl-L-2-amino-8-oxodecanoyl)] (Darkin-Rattray et al., Proc. Natl. Acad. Sci. USA 93, 13143-13147 (1996)); Apicidin Ia, Apicidin Ib, Apicidin Ic, Apicidin IIa, and Apicidin IIb (P. Dulski et al., PCT Application WO 97/11366); CHAP, HC-toxin cyclic tetrapeptide (Bosch et al., Plant Cell 7, 1941-1950 (1995)); WF27082 cyclic tetrapeptide (PCT Application WO 98/48825); and Chlamydocin (Bosch et al., supra).

C. Short chain fatty acid (SCFA) derivatives such as: Sodium Butyrate (Cousens et al., J. Biol. Chem. 254, 1716-1723 (1979)); Isovalerate (McBain et al., Biochem. Pharm. 53: 1357-1368 (1997)); Valerate (McBain et al., supra); 4-Phenylbutyrate (4-PBA) (Lea and Tulsyan, Anticancer Research, 15, 879-873 (1995)); Phenylbutyrate (PB) (Wang et al., Cancer Research, 59, 2766-2799 (1999)); Propionate (McBain et al., supra); Butyramide (Lea and Tulsyan, supra); Isobutyramide (Lea and Tulsyan, supra); Phenylacetate (Lea and Tulsyan, supra); 3-Bromopropionate (Lea and Tulsyan, supra); Tributyrin (Guan et al., Cancer Research, 60, 749-755 (2000)); Valproic acid, Valproate, and Pivanex™.

D. Benzamide derivatives such as CI-994; MS-275 [N-(2-aminophenyl)-4-[N-(pyridin-3-yl methoxycarbonyl)aminomethyl]benzamide] (Saito et al., Proc. Natl. Acad. Sci. USA 96, 4592-4597 (1999)); and 3′-amino derivative of MS-275 (Saito et al., supra).

E. Electrophilic ketone derivatives such as Trifluoromethyl ketones (Frey et al, Bioorganic & Med. Chem. Lett., 12, 3443-3447 (2002); U.S. Pat. No. 6,511,990) and α-keto amides such as N-methyl-α-ketoamides.

F. Other HDAC Inhibitors such as natural products, psammaplins, and Depudecin (Kwon et al. PNAS 95: 3356-3361 (1998)).

HDAC inhibitors include those disclosed in U.S. Pat. Nos. 5,369,108, 5,932,616, 5,700,811, 6,087,367, and 6,511,990, issued to some of the present inventors, the entire contents of which are incorporated herein by reference, non-limiting examples of which are set forth below:

Specific HDAC inhibitors include suberoylanilide hydroxamic acid (SAHA; N-Hydroxy-N′-phenyl octanediamide), which is represented by the following structural formula:

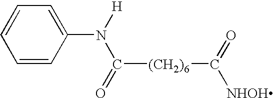

Other examples of such compounds and other HDAC inhibitors can be found in U.S. Pat. No. 5,369,108, issued on Nov. 29, 1994, U.S. Pat. No. 5,700,811, issued on Dec. 23, 1997, U.S. Pat. No. 5,773,474, issued on Jun. 30, 1998, U.S. Pat. No. 5,932,616, issued on Aug. 3, 1999 and U.S. Pat. No. 6,511,990, issued Jan. 28, 2003, all to Breslow et al.; U.S. Pat. No. 5,055,608, issued on Oct. 8, 1991, U.S. Pat. No. 5,175,191, issued on Dec. 29, 1992 and U.S. Pat. No. 5,608,108, issued on Mar. 4, 1997, all to Marks et al.; as well as Yoshida, M., et al., Bioassays 17, 423-430 (1995); Saito, A., et al., PNAS USA 96, 4592-4597, (1999); Furamai R. et al., PNAS USA 98 (1), 87-92 (2001); Komatsu, Y., et al., Cancer Res. 61(11), 4459-4466 (2001); Su, G. H., et al., Cancer Res. 60, 3137-3142 (2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 to Sloan-Kettering Institute for Cancer Research and The Trustees of Columbia University; published PCT Application WO 02/246144 to Hoffmann-La Roche; published PCT Application WO 02/22577 to Novartis; published PCT Application WO 02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; Curtin M. (Current patent status of HDAC inhibitors Expert Opin. Ther. Patents 12(9): 1375-1384 (2002) and references cited therein); and U.S. patent application Ser. Nos. 10/600,132 (Publication No. 20040122101, filed Jun. 19, 2003) and 11/981,367 (filed Oct. 30, 2007).

SAHA or any of the other HDACs can be synthesized according to the method set forth in U.S. Pat. Nos. 5,369,108, 5,700,811, 5,932,616 and 6,511,990, the contents of which are incorporated by reference in their entirety, or according to any other method known to a person skilled in the art.

Specific non-limiting examples of HDAC inhibitors are provided in the Table A below. It should be noted that the present invention encompasses any compounds which are structurally similar to the compounds represented below, and which are capable of inhibiting histone deacetylases.

TABLE A

| Name | Structure |
|---|---|
| MS-275 | 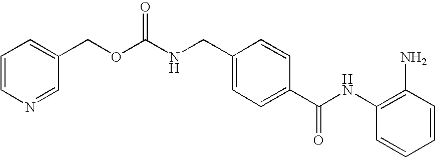 |
| DEPSIPEPTIDE | 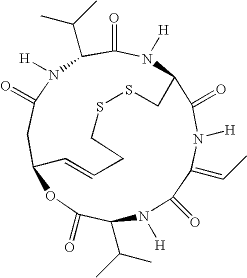 |
| CI-994 | 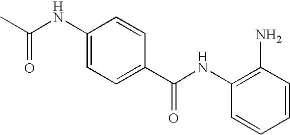 |
| Apicidin | 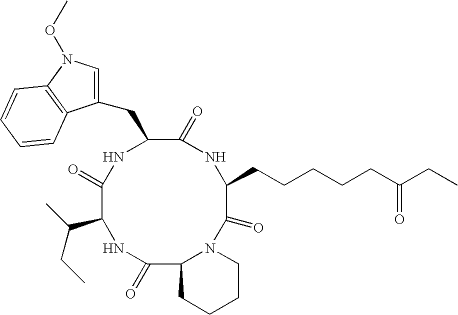 |
| A-161906 | 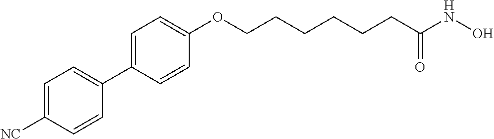 |
| Scriptaid | 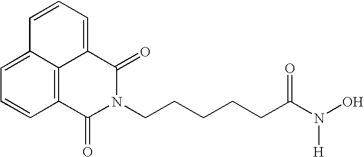 |
| PXD-101 | 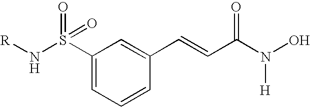 |
| CHAP | 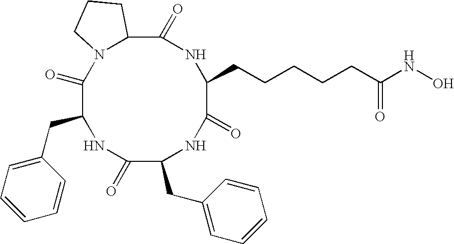 |
| LAQ-824 | 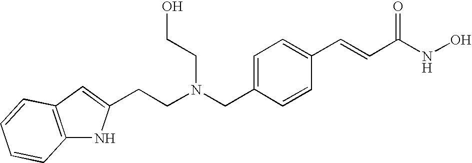 |
| Butyric Acid | 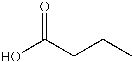 |
| Depudecin | 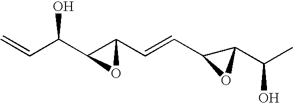 |
| Oxamflatin | 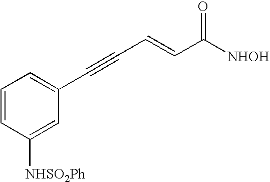 |
| Trichostatin C | 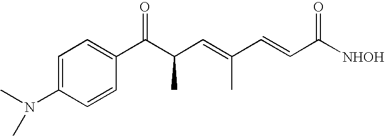 |

### Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the HDAC inhibitors of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, for example by formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least 99% enantiomeric excess.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

This invention is also intended, but not limited to, encompass pro-drugs of the HDAC inhibitors disclosed herein. A prodrug of any of the compounds can be made using well known pharmacological techniques.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

### Anti-Viral Agents

In this invention, SAHA is used in combination with one or more anti-retroviral agents, including: (1) nucleoside reverse transcriptase inhibitors, (2) non-nucleoside reverse transcriptase inhibitors, (3) protease inhibitors, (4) virus uptake/adsorption inhibitors, (5) virus receptor antagonists, (6) viral fusion inhibitors, (7) viral integrase inhibitors, and (8) transcription inhibitors, or other anti-retroviral agents used in treatment of HIV infection.

In a preferred embodiment, the one or more anti-retroviral agents are reverse transcriptase inhibitors. In one aspect, the inhibitors are nucleoside/nucleotide reverse transcriptase inhibitors, which are nucleoside or nucleotide analogs that inhibit action of the viral reverse transcriptase required for conversion of the viral RNA into DNA during viral replication. These inhibitors include, without limitation, azidothymidine and its derivatives (e.g., AZT, Zidovudine), (2R,cis)-4-amino-1-(2-hydroxymethyl-1-1-oxathiolan-5-yl)-(1H)-pyrimidine-2-one (i.e., Lamivudine), 2′,3′-dideoxyinosine (didanosine), 2′,3′-dideoxycytidine (i.e., Zalcitabine), 2′,3′-didehydro-3′-deoxythymidine (i.e., stavudine), (1S,cis)-4-[2-amino-6-(cyclopropylamino)-9H-purin-9-yl]-2-cyclopentene-1-methanol sulfate (i.e., abacavir), (−)-beta-2′,3′-dideoxy-5-fluoro-3′-thiacytidine (i.e., emtricitabine), and phosphonate 9-R-(2-phosphonomethoxypropyl) adenine (i.e., PMPA; tenofovir disoproxil fumarate; adefovir) and various derivatives thereof (see for example, Deeks, S. G. et al., Antimicrob. Agents Chemother. 42(9):2380 2384 (1998)).

In another aspect, the one or more antiviral agents are non-nucleoside reverse transcriptase inhibitors (NNRTI). These agents also inhibit the action of viral reverse transcriptase by binding to the enzyme and disrupting its catalytic activity. Inhibitors include, but are not limited to, 11-cyclopropyl-5,11-dihydro-4-methyl-6H-dipyrido-[3,3-b-2′,3′-][1,4]diaze-pin-6-one (i.e., nevirapine); piperazine,1-[3-[(1-methyl-ethyl)amino]-2-pyridinyl]-4-[[5-(methylsulfonyl)amino]-1-H-indol-2-yl]carbonyl]-monomethane sulfonate (i.e., Delavirdine); and (S)-6-chloro-4-(cyclopropylethynyl)-1,4-dihydro-4-(trifluoromethyl)-2H-3,1-benzoxazine-2-one (i.e., efavirenz). Other include quinazolinone and it derivatives, for example trifluoromethyl-containing quinazolin-2(1-H)-ones (Corbett, J. W. et al., Prog. Med. Chem. 40:63 105 (2000)); calanolide A (Newman, R. A. et al. J. Pharm. Sci. 87(9):1077 1080 (1998)); and 6-arylmethyl-1-(ethoxymethyl)-5-alkyluracil (i.e., emivirine) and its analogs (see El-Brollosy, N. R., J. Med. Chem. 45(26):5721 5726 (2002)).

In one aspect of the invention, SAHA is administered with efavirenz, a non-nucleoside reverse transcriptase (RT) inhibitor of human immunodeficiency virus type 1 (HIV-1). Efavirenz activity is mediated predominantly by noncompetitive inhibition of HIV-1 RT. HIV-2 RT and human cellular DNA polymerases alpha, beta, gamma, and delta are not inhibited by efavirenz.

Efavirenz is chemically described as (S)-6-chloro-4-(cyclopropylethynyl)-1,4-dihydro-4-(trifluoromethyl)-2H-3,1-benzoxazin-2-one and its empirical formula is C₁₄4H₉ClF₃NO₂. Efavirenz is a white to slightly pink crystalline powder with a molecular mass of 315.68. It is practically insoluble in water.

Efavirenz, SUSTIVA® in combination with other antiretroviral agents is indicated for the treatment of HIV-1 infection. This indication is based on two clinical trials of at least one year duration that demonstrated prolonged suppression of HIV-RNA. SUSTIVA® is available as capsules for oral administration containing either 50 mg, 100 mg, or 200 mg of efavirenz and the following inactive ingredients: lactose monohydrate, magnesium stearate, sodium lauryl sulfate, and sodium starch glycolate. The capsule shell contains the following inactive ingredients and dyes: gelatin, sodium lauryl sulfate, titanium dioxide and/or yellow iron oxide. The capsule shells may also contain silicon dioxide. SUSTIVA is also available as film-coated tablets for oral administration containing 600 mg of efavirenz and the following inactive ingredients: croscarmellose sodium, hydroxypropyl cellulose, lactose monohydrate, magnesium stearate, microcrystalline cellulose, and sodium lauryl sulfate. The film coating contains OpadryR Yellow and OpadryR Clear. The tablets are polished with carnauba wax and printed with purple ink, OpacodeR WB.

In a further aspect, the one or more antiviral agents are protease inhibitors. Without being bound by theory, protease inhibitors appear to inhibit HIV replication at the postintegrational level after the virus is integrated into the host chromosome. The target HIV protease enzyme, a 99-amino acid homodimer, cleaves pol-gag polypeptides on the viral envelope. The gag-pol precursor contains the amino acid sequences of various HIV proteins, such as proteins that form the capsid (p19) and nucleocapsid (p24). In addition, gag-pol also contains the sequence of retroviral enzymes, such as reverse transcriptase, proteases, and integrase Inhibition of the HIV protease results in release of immature, noninfectious viral particles. Many of the protease inhibitors may also exert additional antiviral effects by inhibiting proteasome function in the cells. Protease inhibitors useful in the present invention include without limitation the agents indinavir, saquinavir (fortovase), ritonavir, nelfinavir, amprenavir, and lopinavir and pharmaceutically acceptable salts thereof.

In one preferred aspect of the invention, SAHA is administered with the protease inhibitor indinavir sulfate, CRIXIVAN®. Indinavir activity is mediated predominantly by inhibiting HIV-1 protease, an enzyme required for the proteolytic cleavage of the viral polyprotein precursors into the individual functional proteins found in infectious HIV-1. Indinavir binds to the protease active site and inhibits the activity of the enzyme. This inhibition prevents cleavage of the viral polyproteins resulting in the formation of immature non-infectious viral particles.

The chemical name for indinavir sulfate is [1(1S,2R),5(S)]-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythro-pentonamidesulfate (1:1) salt. Indinavir sulfate is a white to off-white, hygroscopic, crystalline powder with the molecular formula C₃₆H₄₇N₅O₄.H₂SO₄ and a molecular weight of 711.88. It is very soluble in water and in methanol.

CRIXIVAN® capsules are formulated as a sulfate salt and are available for oral administration in strengths of 100, 200, 333, and 400 mg of indinavir (corresponding to 125, 250, 416.3, and 500 mg indinavir sulfate, respectively). Each capsule also contains the inactive ingredients anhydrous lactose and magnesium stearate. The capsule shell has the following inactive ingredients and dyes: gelatin, titanium dioxide, silicon dioxide and sodium lauryl sulfate.

In certain embodiments, SAHA may be administered in combination with an integrase inhibitor. Virus replication may be affected by inhibiting the action of integrase, a viral protein involved in inserting the human immunodeficiency virus type 1 (HIV-1) proviral DNA into the host genome. This class of inhibitors may comprise small molecule inhibitors or peptide inhibitors. Small molecule inhibitors, include, among others, integramycin (Singh, S. B. et al, Org. Lett. 4(7):1123 1126 (2002)); diketo derivatives (Vandegraaff, N. et al., Antimicrob. Agents Chemother. 45(9):2510 2516 (2001)); polyhydroxylated styrylquinolines (Zouhiri, F. et al., J. Med. Chem. 43(8):1533 1540 (2000)); and cyclodidemniserinol trisulfate (Mitchell, S. S. et al., Org. Lett. 2(11):1605 1607 (2000)). Peptide based inhibitors include, among others, linear peptides (Puras Lutzke R. A. et al., Proc. Natl. Acad. Sci. USA 92(25):11456 11460 (1995)); (de Soultrait V. R. et al., J Mol. Biol. 342 (2):195-213 (2002)); cyclic peptides (Singh, S. B. et al., J. Nat. Prod. 64(7):874 882 (2001)); and antibodies that bind and inhibit integrase activity (Yi, J. et al., J. Biol. Chem. 277(14):12164 12174 (2002)).

In another preferred aspect of the invention, SAHA is administered with integrase inhibitor MK-0518, raltegravir potassium, N-[(4-Fluorophenyl)methyl]-1,6-dihydro-5-hydroxy-1-methyl-2-[1-methyl-1-[[(5-methyl-1,3,4-oxadiazol-2-1)carbonyl]amino]ethyl]-6-oxo-4-pyrimidine-carboxamide monopotassium salt, C₂₀H₂₀FKN₆O₅, mol wt 482.51, which has the following chemical formula:

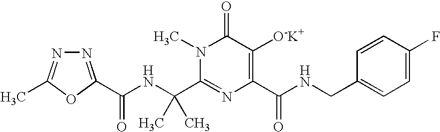

MK-0518, an investigational medicine currently in Phase III, has been shown to block HIV integration into the host genome. MK-0518 had been tested by oral administration, twice daily at doses 200, 400 and 600 mg. See, for example, U.S. Pat. No. 7,169,780.

In certain embodiments, SAHA can be administered with a viral entry inhibitor. Additional antiviral agents useful in the present invention include compounds that inhibit or reduce entry of the virus into the cell. Some virus absorption inhibitors, such as cosalane derivatives, inhibit both the binding of gp120 to CD4 and fusion of the virus with the cell. Other agents inhibit fusion and/or absorption of the viral envelope with the cell membrane and include, among others, pentafuside (T-20); T-1249, a derivative of T-20; and betulinic acid. In another aspect, the inhibitors of viral entry are antagonists of viral co-receptors CXCR4 and CCR5, the 7-transmembrane-domain chemokine receptors used by all HIV-1 strains to infect cells. Interaction of viral protein gp120 with CD4 renders Env competent to bind the co-receptors. It is known that HIV-1 strain designated as R5 uses CCR5 receptors; strains X4 use CXCR4 receptors and R5X4 strains use both chemokine receptors. Viruses that successfully establish infections in previously uninfected hosts are generally R5 virus strains while emergence of X4 is correlated with accelerated disease progression. Agents capable of blockading interaction of viral Env with co-receptor CXCR4 include, bicyclam derivatives (Dessolin, J. et al., J. Med. Chem. 42(2):229 241 (1999)); peptide inhibitors, for example ([Tyr-5,12,Lys7]-polyphemusin II) and its analogs, (Murakami, T. et al., J. Exp. Med. 186(8):1389 1393 (1997) and Arakaki, R. et al., J. Virol. 73(2):1719 23 (1999)) and N-alpha-acetyl-nona-D-arginine (Arg) amide (Doranz, B. J. et al., J. Exp. Med. 186(8):1395 1400 (1997); and distamycin analogs, 2,214,4′-[[aminocarbonyl]amino]bis[-di[pyrrole-2-carboxamide-1,1′-dimethyl]]-6,8 napthalenedisulfonic acid]hexasodium salt (Howard, O. M. et al., J. Leukoc. Biol. 64(1): 6 13 (1998)). Agents known to block interaction of virus with CCR5, include, among others, 1-[(2,4-dimethyl-3-pyridinyl)carbonyl]-4-methyl-4-[3(S)-methyl-4-[1(S)-[4-(trifluoromethyl)phenyl]ethyl]-1-piperazinyl]-piperidine N1-oxide (Tagat, J. R. J. Med. Chem. 44(21):3343 3346 (2001)); SCH—C (Strizki, J. M. et al., Proc. Natl. Acad. Sci. USA 98(22):12718 12723 (2001)); TAK-779 (Baba, M. et al., Proc. Natl. Acad. Sci. USA 96: 5698 5703 (1999)); and antibodies to CCR5 (Simmons, G. et al., Science 276: 279 (1997)); Auraro, S. et al., J. Virol. 74:4402 4406 (2001)).

In other embodiments, SAHA may be administered in combination with inhibitors of viral transcription and/or cell cycle inhibitors. The anti-retroviral agents may also comprise agents directed at inhibition of viral specific transcription or cell cycle inhibitors. One type of viral specific transcription inhibitor is a bistriazoloacridone analog (i.e., temacrazine (1,4-bis[3-(6-oxo-6H-v-triazolo[4,5,1-de]acridin-5-yl)amino-propyl]piperazine) and is described in Turpin, J. A. et al. Antimicrob. Agents Chemother. 42(3):487 494 (1998), hereby incorporated by reference. Inhibitors of the cell cycle are also known to inhibit viral replication, and include, among others, flavopiridol and roscovitine, both of which act by inhibiting cyclin dependent kinases.

In view of the known efficacy of multi-drug combinations of various antiviral compounds, also known in the art as “drug cocktails,” encompassed with the scope of the invention are compositions comprising multiple drug combinations including SAHA. HAART (Highly Active Anti-retroviral Therapy) is a drug regimen consisting of at least three different anti-retroviral drugs. Thus, a plurality of anti-retroviral agents may be used in the present invention. These multi-drug combinations, include, but are not limited to, combinations of the various classes of antiviral agents described above.

Specific non-limiting examples of antiviral for use in combination with the HDAC inhibitor compounds of the present invention include, but are not limited, for example to those listed in Table B as follows:

TABLE B

| NAME | TYPE |
|---|---|
| abacavir, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, ZDU, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, Rescriptor ® | nnRTI |
| efavirenz, Sustiva ® , Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enteric coated didanosine, Videx EC ® | nRTI |
| enfuvirtide, Fuzeon ® | FI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| Lopinavir | PI |
| lopinavir + ritonavir, Kaletra ® | PI |
| nelfinavir, Viracept ® | PI |
| nevirapine, Viramune ® | nnRTI |
| PPL-100 (also known as PL-462) (Ambrilia) | PI |
| ritonavir, Norvir ® | PI |
| raltegravir, MK-518 | InI |
| saquinavir, Invirase ® , Fortovase ® | PI |
| stavudine, d4T,didehydrodeoxythymidine, Zerit ® | nRTI |
| tenofovir DF (DF = disoproxil fumarate), Viread ® | nRTI |
| tipranavir, Aptivus ® | PI |

FI = fusion inhibitor
InI = integrase inhibitor
PI = protease inhibitor
nRTI = nucleoside reverse transcriptase inhibitor
nnRTI = non-nucleoside reverse transcriptase inhibitor
Some of the drugs listed in the table are used in a salt form; e.g., indinavir sulfate, atazanvir sulfate, nelfinvavir mesylate.

### Administration of the HDAC Inhibitor

### Routes of Administration

The HDAC inhibitor (e.g. SAHA), can be administered by any known administration method known to a person skilled in the art. Examples of routes of administration include but are not limited to oral, parenteral, intraperitoneal, intravenous, intraarterial, transdermal, topical, sublingual, intramuscular, rectal, transbuccal, intranasal, liposomal, via inhalation, vaginal, intraoccular, via local delivery by catheter or stent, subcutaneous, intraadiposal, intraarticular, intrathecal, or in a slow release dosage form. SAHA or any one of the HDAC inhibitors can be administered in accordance with any dose and dosing schedule that, together with the effect of the anti-viral agent, achieves a dose effective to treat HIV infection.

Of course, the route of administration of SAHA or any one of the other HDAC inhibitors is independent of the route of administration of the anti-viral agent. A particular route of administration for SAHA is oral administration. Thus, in accordance with this embodiment, SAHA is administered orally, and the second agent (anti-viral) can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

As examples, the HDAC inhibitors, (e.g. SAHA) of the invention can be administered in such oral forms as tablets, capsules (each of which includes sustained release or timed release formulations), pills, powders, granules, elixirs, tinctures, suspensions, syrups, and emulsions. Likewise, the HDAC inhibitors, (e.g. SAHA) can be administered by intravenous (e.g., bolus or infusion), intraperitoneal, subcutaneous, intramuscular, or other routes using forms well known to those of ordinary skill in the pharmaceutical arts. A particular route of administration of the HDAC inhibitor, (e.g. SAHA) is oral administration.

The HDAC inhibitors, (e.g. SAHA) can also be administered in the form of a depot injection or implant preparation, which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The HDAC inhibitor, (e.g. SAHA) can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines. Liposomal preparations of tyrosine kinase inhibitors may also be used in the methods of the invention. Liposome versions of tyrosine kinase inhibitors may be used to increase tolerance to the inhibitors.

The HDAC inhibitors, (e.g. SAHA), can also be delivered by the use of monoclonal antibodies as individual carriers to which the compound molecules are coupled.

The HDAC inhibitors, (e.g. SAHA), can also be prepared with soluble polymers as targetable drug carriers. Such polymers can include polyvinyl pyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the HDAC inhibitors can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

In a specific embodiment, the HDAC inhibitor, e.g. SAHA, is administered orally in a gelatin capsule, which can comprise excipients such as microcrystalline cellulose, croscarmellose sodium and magnesium stearate.

Dosages and Dosage Schedules

The dosage regimen utilizing the HDAC inhibitors can be selected in accordance with a variety of factors including type, species, age, weight, sex and the type of disease being treated; the severity (i.e., stage) of the disease to be treated; the route of administration; the renal and hepatic function of the patient; and the particular compound or salt thereof employed. A dosage regimen can be used, for example, to prevent, inhibit (fully or partially), or arrest the progress of the disease.

In accordance with the invention, an HDAC inhibitor (e.g., SAHA or a pharmaceutically acceptable salt or hydrate thereof) can be administered by continuous or intermittent dosages. For example, intermittent administration of an HDAC inhibitor may be administration one to six days per week or it may mean administration in cycles (e.g. daily administration for two to eight consecutive weeks, then a rest period with no administration for up to one week) or it may mean administration on alternate days. The compositions may be administered in cycles, with rest periods in between the cycles (e.g. treatment for two to eight weeks with a rest period of up to a week between treatments).

For example, SAHA or any one of the HDAC inhibitors can be administered in a total daily dose of up to 800 mg. The HDAC inhibitor can be administered once daily (QD), or divided into multiple daily doses such as twice daily (BID), and three times daily (TID). The HDAC inhibitor can be administered at a total daily dosage of up to 800 mg, e.g., 200 mg, 300 mg, 400 mg, 600 mg, or 800 mg, which can be administered in one daily dose or can be divided into multiple daily doses as described above. In specific aspects, the administration is oral.

Intravenously or subcutaneously, the patient would receive the HDAC inhibitor in quantities sufficient to deliver between about 3-1500 mg/m² per day, for example, about 3, 30, 60, 90, 180, 300, 600, 900, 1200 or 1500 mg/m² per day. Such quantities may be administered in a number of suitable ways, e.g. large volumes of low concentrations of HDAC inhibitor during one extended period of time or several times a day. The quantities can be administered for one or more consecutive days, intermittent days or a combination thereof per week (7-day period). Alternatively, low volumes of high concentrations of HDAC inhibitor during a short period of time, e.g. once a day for one or more days either consecutively, intermittently or a combination thereof per week (7-day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/m² total treatment.

Typically, an intravenous formulation may be prepared which contains a concentration of HDAC inhibitor of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

Subcutaneous formulations can be prepared according to procedures well known in the art at a pH in the range between about 5 and about 12, which include suitable buffers and isotonicity agents, as described below. They can be formulated to deliver a daily dose of HDAC inhibitor in one or more daily subcutaneous administrations, e.g., one, two or three times each day.

The various modes of administration, dosages, and dosing schedules described herein merely set forth specific embodiments and should not be construed as limiting the broad scope of the invention. Any permutations, variations, and combinations of the dosages and dosing schedules are included within the scope of the present invention.

### Administration of Anti-Viral Agents.

The anti-viral agents of the present invention may be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The anti-viral compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions comprising anti-viral agents of the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21^{st} Edition, Lippincott Williams & Wilkins, 2005.

The anti-viral agents of this invention can be administered orally in a dosage range of about 0.001 to about 1000 mg/kg of mammal (e.g., human) body weight per day in a single dose or in divided doses. One preferred dosage range is about 0.01 to about 500 mg/kg body weight per day orally in a single dose or in divided doses. Another preferred dosage range is about 0.1 to about 100 mg/kg body weight per day orally in single or divided doses. For oral administration, the compositions can be provided in the form of tablets or capsules containing about 1.0 to about 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. The anti-viral agents of the invention can also be administered according to known procedures, known dosages, and known dosing regimens. Further description of routes of administration, dosages, and dosage forms for anti-retroviral agents of the present invention is provided in The Merck Manual of Therapy and Diagnosis, 18^{th} Edition, John Wiley & Sons, New York, N.Y.

### Combination Administration.

In accordance with the invention, any one or more of the specific dosages and dosage schedules of the HDAC inhibitors are also applicable to any one or more of the anti-retroviral agents to be used in the combination treatment.

In various aspects of the invention, the treatment procedures are performed sequentially in any order, concurrently, consequently, alternatively or a combination thereof. For example, the first treatment procedure, e.g., administration of an HDAC inhibitor, can take place prior to the second treatment procedure, e.g., the anti-viral agent, after the second treatment with the anti-viral agent, at the same time as the second treatment with the anti-viral, or a combination thereof.

In one aspect of the invention, a total treatment period can be decided for the HDAC inhibitor. The anti-viral agent can be administered prior to onset of treatment with the HDAC inhibitor or following treatment with the HDAC inhibitor. In addition, the anti-viral agent can be administered during the period of HDAC inhibitor administration but does not need to occur over the entire HDAC inhibitor treatment period. Similarly, the HDAC inhibitor can be administered prior to onset of treatment with the anti-viral agent or following treatment with the anti-viral agent. In addition, the HDAC inhibitor can be administered during the period of anti-viral agent administration but does not need to occur over the entire anti-viral agent treatment period.

Pharmaceutical Compositions

As described above, the compositions comprising the HDAC inhibitor and/or the anti-viral agent can be formulated in any dosage form suitable for oral, parenteral, intraperitoneal, intravenous, intraarterial, transdermal, sublingual, intramuscular, rectal, transbuccal, intranasal, liposomal, via inhalation, vaginal, or intraocular administration, for administration via local delivery by catheter or stent, or for subcutaneous, intraadiposal, intraarticular, intrathecal administration, or for administration in a slow release dosage form.

The HDAC inhibitor and the anti-viral agent can be formulated in the same formulation for simultaneous administration, or they can be in two separate dosage forms, which may be administered simultaneously or sequentially as described above.

The invention also encompasses pharmaceutical compositions comprising pharmaceutically acceptable salts of the HDAC inhibitors and/or the anti-viral agents.

Suitable pharmaceutically acceptable salts of the compounds described herein and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.); an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

The invention also encompasses pharmaceutical compositions comprising hydrates of the HDAC inhibitors and/or the anti-viral agents.

In addition, this invention also encompasses pharmaceutical compositions comprising any solid or liquid physical form of SAHA or any of the other HDAC inhibitors. For example, The HDAC inhibitors can be in a crystalline form, in amorphous form, and have any particle size. The HDAC inhibitor particles may be micronized, or may be agglomerated, particulate granules, powders, oils, oily suspensions or any other form of solid or liquid physical form.

For oral administration, the pharmaceutical compositions can be liquid or solid. Suitable solid oral formulations include tablets, capsules, pills, granules, pellets, and the like. Suitable liquid oral formulations include solutions, suspensions, dispersions, emulsions, oils, and the like.

Any inert excipient that is commonly used as a carrier or diluent may be used in the formulations of the present invention, such as for example, a gum, a starch, a sugar, a cellulosic material, an acrylate, or mixtures thereof. The compositions may further comprise a disintegrating agent and a lubricant, and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

The HDAC inhibitors can be administered as active ingredients in admixture with suitable pharmaceutical diluents, excipients or carriers (collectively referred to herein as “carrier” materials or “pharmaceutically acceptable carriers”) suitably selected with respect to the intended form of administration. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions, or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic, or oily solutions and the like as detailed above.

Subcutaneous formulations can be prepared according to procedures well known in the art at a pH in the range between about 5 and about 12, which include suitable buffers and isotonicity agents. They can be formulated to deliver a daily dose of the active agent in one or more daily subcutaneous administrations. The choice of appropriate buffer and pH of a formulation, depending on solubility of the HDAC inhibitor to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A particular pH range for subcutaneous formulation of an HDAC inhibitor a hydroxamic acid moiety can be about 9 to about 12.

The compositions of the present invention can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

### EXAMPLES

The examples are presented in order to more fully illustrate the various embodiments of the invention. These examples should in no way be construed as limiting the scope of the invention recited in the appended claims.

### Example 1

### Expression of Latent HIV is Induced by SAHA

These experiments have investigated the ability of HDAC inhibitor, such as SAHA, to induce HIV promoter expression and virus production from resting CD4⁺ cells. Specifically, the effect of SAHA on J89, a Jurkat T cell line infected with a single HIV genome encoding the enhanced green fluorescence protein (EGFP) was characterized.

For these experiments, J89 cells (2×10⁶ cells) were washed with phosphate-buffered saline (PBS) and incubated overnight at 37° C. under 5% carbon dioxide in media containing 0.5% fetal bovine serum (FBS; Invitrogen, Carlsbad, Calif., USA). Cells were then washed and incubated for 2 hours in media without serum and containing 400 nmol/1 trichostatin A (Sigma, St. Louis, Mo., USA) or 1-5 mmol/1 VPA (Sigma), or they were untreated. FBS was then added to a final concentration of 20% and cells were incubated only for an additional 2 hours to avoid the induction of secondary gene effects. Cells were washed with PBS, cross-linked with 1% formaldehyde, washed again, snap-frozen in an ethanol/dry-ice bath and stored at −80° C.

Further, histone acetylation at nucleosome 1 of the HIV promoter was assayed by chromatin immunoprecipitation. The chromatin immunoprecipitation assays were carried out as follows: cells were thawed on ice and incubated with 200 μl lysis buffer (Upstate, Waltham, Mass., USA) containing 40 μl protease inhibitor cocktail (Sigma) for 10 minutes at 4° C.; 300 μl dilution buffer (Upstate) was added to the cell lysates, which were then sonicated to fragment chromatin to 500 to 1000 base pairs. After centrifugation of sonicated cell lysates, 60 μg soluble chromatin was incubated overnight with 5 μl antiacetylhistone H4 (Upstate) or rabbit pre-immune immunoglobulin G (Sigma). Immunoprecipitates were incubated with 50 μl salmon sperm DNA/protein A agarose beads (Upstate) for 1 hour at 4° C. Agarose beads were recovered by centrifugation and washed using buffers from the chromatin immunoprecipitation assay kit (Upstate) following the manufacturer's instructions. Further, the EGFP mRNA expression was monitored by RT-PCR and p24 antigen production by ELISA.

Further, limiting-dilution outgrowth assays compared the ability of SAHA, valproic acid, (VPA), and maximal mitogen stimulation (PHA/allo) to induce virus expression from the resting CD4⁺ T cells obtained at four occasions from three aviremic, ART-treated HIV⁺ patients. For these experiments, informed consent was obtained from all patients. Resting CD4⁺ T cells were negatively selected from leukopheresis samples using magnetic bead elution. In limiting dilution format, 5.0−0.1×10⁶ resting CD4⁺ cells were treated with 40 μM VPA, 250 nM SAHA and PHA/allo, respectively, to induce HIV viral expression. The HIV virus was detected by p24 antigen capture ELISA.

As summarized in Table 1, SAHA application efficiently induced chromatin remodeling at nucleosome 1, HIV transcription, virus production in the J89 cell line and virus outgrowth ex vivo from patients' cells.

TABLE 1

| Infected resting cells per billion | Patient | | | |
|---|---|---|---|---|
| after exposure to: | A₁ | A₂ | B | C |
| VPA 40 μM | 223 | n.d. | >11,300 | 25 |
| SAHA 250 nM | 410 | 561 | 1,927 | <31 |
| PHA/allo | 630 | 487 | 17,950 | 41 |

These results implicate SAHA as an excellent and specific target for antilatency therapies. The combination of anti-retroviral therapy (ART) with a potent and selective HDAC inhibitor, such as for example SAHA, may allow effective targeting of persistent viral HIV infection.

### Example 2

### Dose-Response of SAHA into ACH-2 Cell Line Model for HIV Latency

These experiments investigated the ability of SAHA to induce HIV promoter expression and virus production from resting CD4⁺ cells. Specifically, the effect of SAHA on ACH-2 cell line model for HIV latency was characterized.

For these experiments, three T-75 flasks of ACH-2 cells were centrifuged at 1000 rpm for 5 minutes at room temperature. The supernatant was aspirated and cells were resuspend in 10 mL media (RPMI 1640 (Invitrogen, 11835-055), 2 mM L-glutamine (Invitrogen, 10082-139), 10% FBS (Invitrogen, 25030-081), 1× penicillin/streptomycin (Invitrogen, 15140-122)). For further testing, cells were plated into 96-well Costar (cat #3799) round bottom plate. To each well, 90 μL of media and 10 μL of SAHA was added. In addition, one column included addition of DMSO as a negative control. Further, SAHA was added at the following final concentrations: 10 μM, 3 μM, 1 0.8 μM, 0.6 μM, 0.5 μM, 0.4 μM, 0.3 μM, 0.2 μM, 0.1 μM, 0.03 μM, 0.01 μM, 0.003 μM. The cells were further incubated for 24 hours at 37° C., 5% CO₂.

Following the incubation, cells were stained for expression of HIV p24 as follows:

1. Stained 10 μL of samples with 10 μL of Trypan Blue and observed under microscope to evaluate compound cytotoxicity.

2 Centrifuged at 1000 rpm at room temperature for 2 minutes to pellet cells using a Sorvall RT 7 tabletop centrifuge. Resuspended cells with 100 μL cold PBS (Cellgro, #21-031-CM) to wash, further re-centrifuge and remove supernatant.

3 Fixed cells with 100 μL per well 4% paraformaldehyde (IC fixation buffer, eBioscience, #00-8222-49) at 4° C. for 20 minutes. Following fixation the cells were centrifuged as above and the supernatant was removed.

4 Cells were washed twice with 200 μL per well of permeabilization buffer of 0.1% saponin, 0.09% sodium azide (eBioscience, #00-8333-56). After second wash, cells were centrifuged, the supernatant was removed, cells were resuspended in 200 μl, permeabilization buffer and incubated at 4° C. for 10 minutes.

5 Diluted anti-p24 antibody 1:75 (FITC-conjugated anti-HIV core, mouse IgG1, KC57-FITC, Beckman Coulter, #6604665) in permeabilization buffer.

6 Added 50 μL antibody mix to wells and incubated in the dark for 30 minutes at 4° C.

7 Centrifuged plate as above and removed supernatant. Washed cells twice with 200 μL per well of permeabilization buffer as above. Resuspended cells in permeabilization buffer diluted 2-fold.

8 Analyzed percentage of cells expressing HIV p24 protein using Guava Easycyte Flow Cytometer.

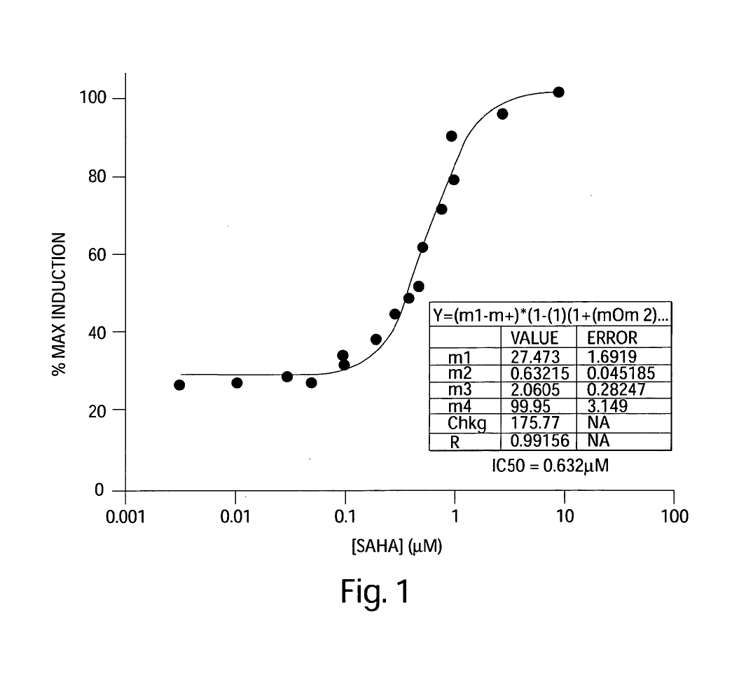

As shown in FIG. 1, SAHA activates expression of HIV in the ACH-2 cell line with an EC₅₀ of 0.632 μM of SAHA, while valproic acid induced 62% of cells with an IC₅₀ of 910 μM, and TSA induced 78% of cells with an IC₅₀ of 98 nM.

### Example 3

### Effectiveness of SAHA in Inducing HIV Expression from Primary Cells Infected with HIV In Vitro

To demonstrate the effectiveness of SAHA (vorinostat) in inducing HIV expression from primary cells infected with HIV in vitro, the following experiment was carried out: CD4⁺ T-cells were purified by using a two-stage process by Biological Specialty (Colmar, Pa.).

Briefly, RosetteSep Human CD4⁺ T-cell enrichment antibody cocktail was added to whole blood in order to cross-link unwanted cells to red blood cells, and CD4⁺ T-cells were collected following centrifugation over Ficoll-Paque PLUS density medium. Enriched CD4⁺ T-cells were furthered purified by negative selection to remove residual CD8⁺ T cells, B cells, monocytes, NK cells, and activated CD4⁺ T cells using appropriate monoclonal antibodies and magnetic beads conjugated with antibodies to mouse immunoglobulin G. The depletion of activated CD4⁺ T cells was accomplished by using antibodies to both early (CD69 and CD25) and late (HLA-DR) activation markers. The resulting populations of resting CD4⁺ T cells showed <1% contamination with activated cells. CD4⁺ T cells were cultured in 10% heat-inactivated human serum in RPMI with 1% penicillin-streptomycin.

HIV infection of the resting CD4⁺ T-cells was accomplished by spinoculating VSVG-pseudotyped R8Δenv/K103N HIV with resting CD4⁺ T cells resuspended at 2×10⁷ cells/ml at 1,200×g for 2 h at 25° C. After spinoculation the cells were washed three times and resuspended in 10% autologous serum in the presence of 1.25 μM saquinavir (Roche US Pharmaceuticals).

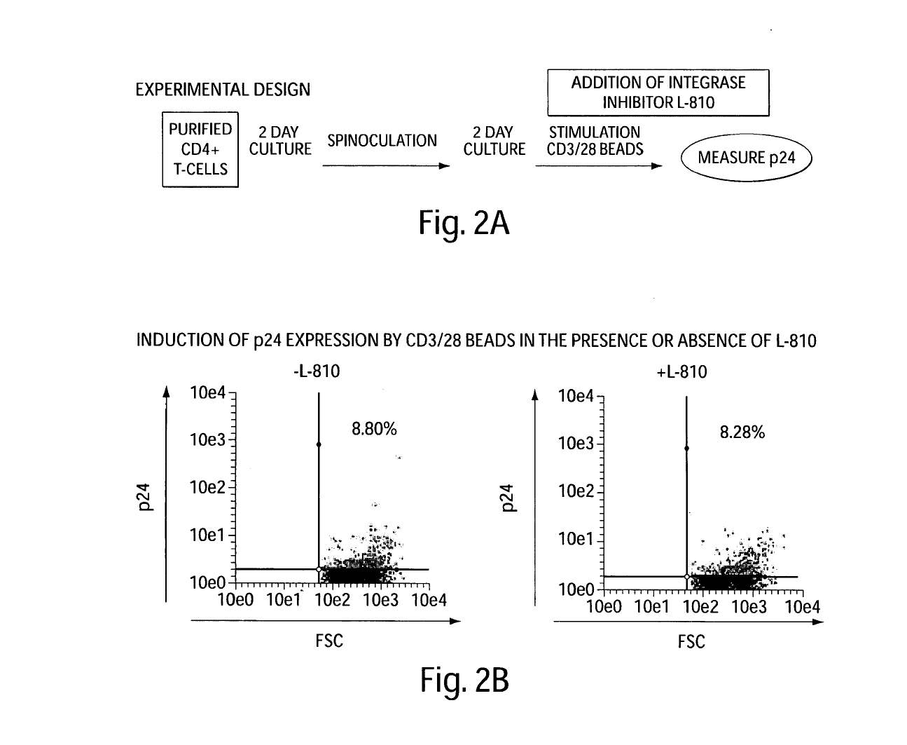

The cells were incubated for two days following spinoculation to allow the completion of reverse transcription and integration of the virus. At this point, the cells were treated with either SAHA (vorinostat), other described LTR activating compounds, or beads coated with anti-CD3 and anti-CD28 (Invitrogen) as a positive control. The anti-CD3/anti-CD28 beads activate expression of HIV by activating the resting cells, and are expected to lead to the expression of all competent integrated virus. The induction of HIV infection was measured by quantifying the percentage of cells expressing Gag by flow cytometry. Cells were fixed with 1% paraformaldehyde for 10 minutes, washed, and stained in permeabilization buffer (2% fetal calf serum, 25 μg/ml murine immunoglobulin G [IgG; BDIS], 0.5% Saponin [Sigma]) with FITC-labeled KC57 (Beckman Coulter Immunology Systems, Miami, Fla.). This IgG1 antibody recognized p55, p39, p33, and p24 proteins of the core antigens of HIV-1. The labeled CD4⁺ resting T-cells were analyzed by flow cytometry using a Guava EasyCyte Flow Cytometer. The experimental design, along with data from a control experiment, is shown in FIGS. 2A and 2B.

FIG. 2B depicts variations in the degree of HIV infection that is inducible by anti-CD3/anti-CD28 beads between experiments carried out in cells obtained from different donors was observed, ranging from 5 to 24% of cells. With a single spinoculation, highly reproducible activation of virus expression by anti-CD3/anti-CD28 beads, as well as a consistent 3-5 fold window between HIV-expressing cells in vehicle-treated (0.1% DMSO) vs. anti-CD3/anti-CD28 bead-treated cells were observed (% p24+; 5-24%+=100% “induction” relative to positive control).

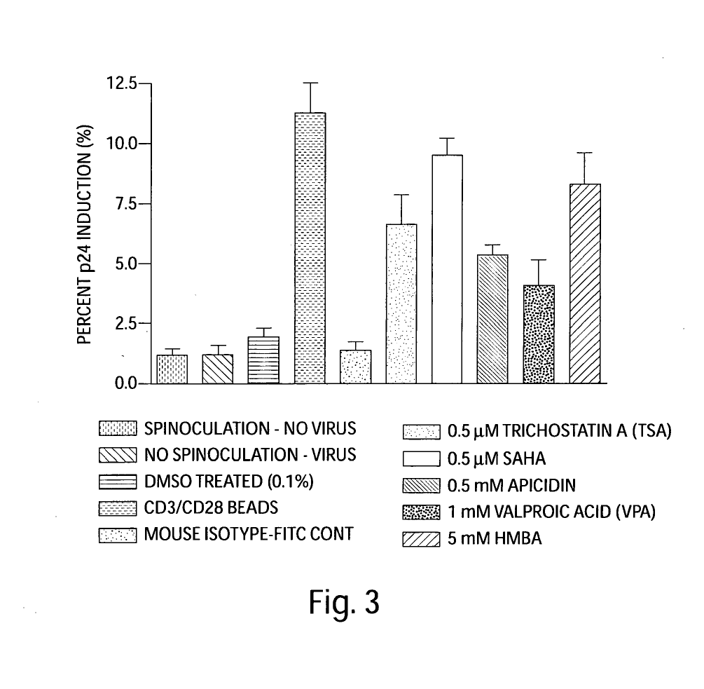

FIG. 3 shows the results of an experiment comparing a number of different HDAC inhibitors, including SAHA (vorinostat), TSA, apicidin, and VPA, and their ability to induce HIV expression from latently resting cells. SAHA (vorinostat) was the most effective inducer of HIV expression with respect to the percentage of cells induced.

What is claimed:

1. A method of treating HIV infection in a subject, said method comprising the step of administering to the subject SAHA (suberoylanilide hydroxamic acid), represented by the structure: 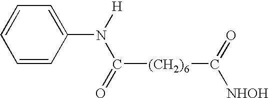
or a pharmaceutically acceptable salt or hydrate thereof and one or more anti-retroviral agents.

2. The method of claim 1, wherein said anti-retroviral agent is selected from the group consisting of a nucleoside reverse transcriptase inhibitor, non-nucleoside reverse transcriptase inhibitor, protease inhibitor, fusion inhibitor, entry inhibitor, integrase inhibitor, co-receptor antagonist, viral adsorption inhibitor, viral specific transcription inhibitor, and cyclin dependent kinase inhibitor and a combination thereof.

3. The method of claim 2, wherein said anti-retroviral agent is selected from the group consisting of a non-nucleoside reverse transcriptase inhibitor, a protease inhibitor, an integrase inhibitor, and a combination thereof.

4. The method of claim 2, wherein said anti-retroviral agent is selected from the group consisting of efavirenz, indinavir sulfate and raltegravir potassium.

5. The method of claim 4, wherein said anti-retroviral agent is raltegravir potassium.

6. A method of depleting latent HIV infection within resting CD4⁺ T cells comprising administering to the subject SAHA (suberoylanilide hydroxamic acid), represented by the structure: 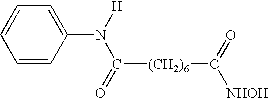
or a pharmaceutically acceptable salt or hydrate thereof.

7. A method of activating expression from the HIV long terminal repeat (LTR) promoter comprising the step of administering to the subject SAHA (suberoylanilide hydroxamic acid), represented by the structure: 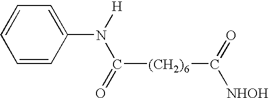
or a pharmaceutically acceptable salt or hydrate thereof.

8. A method of treating latent HIV infection in a subject, said method comprising the step of administering to the subject SAHA (suberoylanilide hydroxamic acid), represented by the structure: 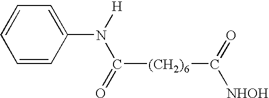
or a pharmaceutically acceptable salt or hydrate thereof and one or more anti-retroviral agents.

9. The method of claim 8, wherein said anti-retroviral agent is selected from the group consisting of a nucleoside reverse transcriptase inhibitor, non-nucleoside reverse transcriptase inhibitor, protease inhibitor, fusion inhibitor, entry inhibitor, integrase inhibitor, co-receptor antagonist, viral adsorption inhibitor, viral specific transcription inhibitor, and cyclin dependent kinase inhibitor and a combination thereof.

10. The method of claim 9, wherein said anti-retroviral agent is selected from the group consisting of a non-nucleoside reverse transcriptase inhibitor, a protease inhibitor, an integrase inhibitor, and a combination thereof.

11. The method of claim 9, wherein said anti-retroviral agent is selected from the group consisting of efavirenz, indinavir sulfate and raltegravir potassium.

12. The method of claim 11, wherein said anti-retroviral agent is raltegravir potassium.

13. The method according to any one of claims 1, 5-8 and 12, wherein SAHA is the active ingredient.

14. A pharmaceutical composition for treating HIV infection in a subject, comprising suberoylanilide hydroxamic acid (SAHA), represented by the structure: 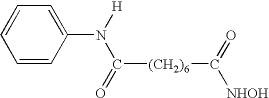
or a pharmaceutically acceptable salt or hydrate thereof in combination with one or more anti-retroviral agents.

15. The pharmaceutical composition of claim 14, wherein said anti-retroviral agent is selected from the group consisting of a nucleoside reverse transcriptase inhibitor, non-nucleoside reverse transcriptase inhibitor, protease inhibitor, fusion inhibitor, entry inhibitor, integrase inhibitor, co-receptor antagonist, viral adsorption inhibitor, viral specific transcription inhibitor, and cyclin dependent kinase inhibitor and a combination thereof.

16. The pharmaceutical composition of claim 15, wherein said anti-retroviral agent is selected from the group consisting of a non-nucleoside reverse transcriptase inhibitor, a protease inhibitor, an integrase inhibitor, and a combination thereof.

17. The pharmaceutical composition of claim 15, wherein said anti-retroviral agent is selected from the group consisting of efavirenz, indinavir sulfate and raltegravir potassium.

18. The pharmaceutical composition of claim 17, wherein said anti-retroviral agent is raltegravir potassium.
